(19) United States
(12) Patent Application Publication
(10) Pub. No.: US 2008/0032920 A1
(43) Pub. Date: Feb. 7, 2008

(54) MACROMOLECULES MODIFIED WITH ELECTROPHILIC GROUPS AND METHODS OF MAKING AND USING THEREOF

(76) Inventors: Glenn D. Prestwich, Salt Lake City, UT (US); Monica Serban, Salt Lake City, UT (US)

Correspondence Address: HELLER EHRMAN LLP, 275 MIDDLEFIELD ROAD, MENLO PARK, CA, 94025-3506, US

(21) Appl. No.: 11/776,526

(22) Filed: Jul. 11, 2007

Related U.S. Application Data
(60) Provisional application No. 60/806,966, filed on Jul. 11, 2006.

(51) Int. Cl. A61K 38/00; A61K 31/12; A61K 31/16; A61P 17/02; C07C 49/04; C07H 21/02; C07K 2/00; C07K 14/00; C07H 1/00; C07C 233/00; A61K 38/16; A61K 31/715

(52) U.S. Cl. 514/2; 514/44; 514/54; 514/616; 514/675; 514/8; 530/395; 536/123.1; 536/23.1; 564/152; 568/382

(57) ABSTRACT

Described herein are macromolecules modified with electrophilic groups and methods of making and using thereof. The preparation of a thiol-reactive, electrophililic derivative of HA in order to prepare “crosslinker-free” hydrogels are described as well as compounds and methods that are capable of coupling two or more molecules, such as macromolecules, under mild conditions. Specifically disclosed is the introduction of reactive bromo- and iodoacetate functionalities at the hydroxyl groups that are abundantly present on the HA polymer. The “crosslinker-free” hydrogels described have numerous applications including, but not limited to, drug delivery, small molecule delivery, wound healing, burn injury healing, tissue regeneration/engineering, cell culturing, and bio-artificial materials.

### RELATED APPLICATIONS

This application claims the benefit of U.S. Provisional Application No. 60/806,966 filed Jul. 11, 2006.

### BACKGROUND

The use of macromolecules in pharmaceutical applications has received considerable attention. At times, it is desirable to couple two or more macromolecules to produce new macromolecule scaffolds with multiple activities. Existing technologies used to couple two or macromolecules, however, present numerous difficulties. For example, the alkaline conditions or high temperatures necessary to create hydrogels with high mechanical strength are cumbersome and harsh. Although the use of crosslinkers to produce macromolecular scaffolds has met with some success, the crosslinking agents are often relatively small, cytotoxic molecules, and the resulting scaffold has to be extracted or washed extensively to remove traces of unreacted reagents and byproducts (Hennink, W. E.; van Nostrum, C. F. Adv. Drug Del. Rev. 2002, 54, 13-36), thus precluding use in many medical applications. A physiologically compatible macromolecular scaffold capable of being produced in a straightforward manner is needed before they will be useful as therapeutic aids.

### SUMMARY

Described herein are macromolecules modified with electrophilic groups and methods of making and using thereof. More specifically, herein described is the preparation of a thiol-reactive, electrophililic derivative of HA in order to prepare “crosslinker-free” hydrogels. Described herein are compounds and methods that are capable of coupling two or more molecules, such as macromolecules, under mild conditions. Specifically disclosed is the introduction of reactive bromo- and iodoacetate functionalities at the hydroxyl groups that are abundantly present on the HA polymer.

The compounds and compositions described herein have numerous applications including, but not limited to, drug delivery, small molecule delivery, wound healing, burn injury healing, tissue regeneration/engineering, cell culturing, and bio-artificial materials.

The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1. Synthetic scheme for bromoacetate derived HA.

FIG. 2. ¹H-NMR spectrum of HA-BA in D₂O.

FIG. 3. Synthesis of iodoacetate derived HA via a Finkelstein reaction.

FIG. 4. ¹H-NMR spectrum of HA-IA in D₂O.

FIG. 5. SAMSA derivatization of hyaluronan derivatives. A—Structure of SAMSA fluorescein. B—Fluorescence of SAMSA derivatized compounds under UV light. C—A_{495 nm} data. D—UV/VIS scan of SAMSA conjugated compounds.

FIG. 6. T31 fibroblast viability in the presence of haloacetate HAs. Black bar—untreated control; white bars—HABA treatment; grey bars—HAIA treatment (*p<0.005, **p <0.5 and ***p>0.05 vs. untreated control). Columns represent mean ±S.D., n=6.

FIG. 7. Schematic depiction of HA haloacetate-containing hydrogels. A. Crosslinking of HA haloacetate with CMHA-S affords non-cytoadhesive hydrogels. B. Crosslinking of HA haloacetate with CMHA-S and Gtn-DTPH affords cytoadhesive hydrogels.

FIG. 8. Viability of fibroblasts cultured on haloacetate HA hydrogels, as determined by MTS colorimetric assay. White bars—hydrogels without Gtn-DTPH; grey bars—hydrogels with Gtn-DTPH (*p<0.001, **p<0.05 and ***p>0.05 versus the control represented with a black bar). Inset—blow-up of A₄₉₀ values for fibroblasts cultured on Gtn-DTPH-free haloacetate HA hydrogels (*p<0.05 versus CMHA-S). Values represented are mean ±S.D., n=6.

FIG. 9. Hydrogel degradation rates in the presence or absence of HAse (225 U/mL). Each data point represents the mean ±S.D., n=3.

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or can not be substituted and that the description includes both unsubstituted lower alkyl and lower alkyl where there is substitution.

“Parts by weight,” of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

A “residue” of a chemical species, refers to the moiety that is the resulting product of the chemical species in a particular reaction scheme or subsequent formulation or chemical product, regardless of whether the moiety is actually obtained from the chemical species. For example, a polysaccharide that contains at least one —OH group can be represented by the formula Y—OH, where Y is the remainder (i.e., residue) of the polysaccharide molecule.

Variables such as R¹-R⁵, A′, A¹, A², G′, L, o, R, R′, X, X′, Y, Y′, and Z used throughout the application are the same variables as previously defined unless stated to the contrary.

The term “alkyl group” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is an alkyl group containing from one to six carbon atoms.

The term “perfluoroalkyl group” or “fluoroalkyl” as used herein means a branched or unbranched saturated hydrocarbon group of 1 to 24 carbon atoms, wherein at least one of the hydrogen atoms is substituted with fluorine. A perfluoroalkyl group may also mean that all hydrogen atoms of the alkyl group are substituted with fluorine.

The term “aryl group” as used herein is any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “halogen” as used herein is fluoride, chloride, bromide or iodide.

The term “polyalkylene group” or “polyalkelenyl group” as used herein is a group having two or more CH₂ groups linked to one another. The polyalkylene group can be represented by the formula —(CH₂)ₙ—, where n is an integer of from 2 to 25.

The term “polyether group” as used herein is a group having the formula —[(CHR)ₙO]ₘ—, where R is hydrogen or a lower alkyl group, n is an integer of from 1 to 20, and m is an integer of from 1 to 100. Examples of polyether groups include, polyethylene oxide, polypropylene oxide and polybutylene oxide.

The term “polythioether group” as used herein is a group having the formula —[(CHR)ₙS]ₘ—, where R is hydrogen or a lower alkyl group, n is an integer of from 1 to 20, and m is an integer of from 1 to 100.

The term “polyamino group” as used herein is a group having the formula —[(CHR)ₙNR]ₘ—, where each R is, independently, hydrogen or a lower alkyl group, n is an integer of from 1 to 20, and m is an integer of from 1 to 100.

The term “polyester group” as used herein is a group that is produced by the reaction between a compound having at least two carboxylic acid groups with a compound having at least two hydroxyl groups.

The term “polyamide group” as used herein is a group that is produced by the reaction between a compound having at least two carboxylic acid groups with a compound having at least two unsubstituted or monosubstituted amino groups.

The phrase “substituted with” (as in “substituted with X”) means that a group such as an alkyl group such as a —CH₃ group wherein one or more of the hydrogen atom is “substituted with” or “replaced by” the group X and forms the group —CH₂—X.

### DETAILED DESCRIPTION OF EMBODIMENTS

Two novel HA derivatives bearing haloacetate groups were synthesized and characterized. Both materials elicit cytotoxic effects at high doses when added to normal cell culture medium. When the two new polymers were allowed to react with CMHA-S, a thiol-modified HA derivative, the hydrogels obtained did not support cell attachment and still elicited mild cytotoxic effects. In contrast, the co-crosslinking of CMHA-S plus Gtn-DTPH with HA haloacetates gave hydrogels that supported cell attachment and growth. Thus, the HA haloacetate platform offers access to both cytoadhesive materials or cytocompatible barriers to cell attachment. Moreover, HA haloacetate-containing hydrogels show slow HAse-mediated degradation rates, which make them suitable for in vivo applications. Taken together, our results suggest the adaptability and potential of the new materials for medical applications, specifically for adhesion prevention and medical device coating.

HABA was prepared directly from HA and bromoacetic anhydride, but the use of iodoacetic anhydride for HAIA was avoided because of competing nucleophilic displacement of iodide by hydroxide under the basic conditions employed. Instead, HAIA was prepared from HABA by simple S_{N}2 substitution of bromide by iodide. As anticipated, the HA haloacetates showed a dose dependent cytotoxic effect and tested with cultured T31 human tracheal scar fibroblasts. These primary human cells are more relevant for in vivo studies, yet still capture the responses of fibroblastic cell lines that are often employed for in vitro biocompatibility and in vitro 3-D cytocompatibility experiments.

The reaction of HA haloacetates with nucleophilic macromolecules affords cytocompatible hydrogels. Depending on the composition, the hydrogels may either prevent or promote cell adherence, spreading and proliferation. However, the prolonged gelation times of HA haloacetate-containing hydrogels make these impractical for most 3-D cell encapsulation protocols. Nevertheless, the hydrogels could be used for pseudo-3-D cultures where cells would be seeded on top of hydrogels.

Similar, chemically-modified HA hydrogels were fully degraded in vitro in 3 days in the presence of high levels of hyaluronidase. The in vivo residence time of those subcutaneously implanted gels, was determined to be more than 2 weeks. By analogy, our degradation data obtained under similar enzymatic conditions can be extrapolated to infer that HA haloacetate-containing hydrogels would have a residence time of approximately 4 weeks. The hyaluronidase concentration used in these in vitro assays was approximately 100-fold higher that the physiological enzyme units (ca. 2.6 U/ml in serum).

One potential application for the non-adherent HA haloacetate hydrogels could be adhesion prevention. Conditions such as bowel obstruction, pelvic pain, even infertility can be the results of undesired post-surgical adhesions. Certain HA hydrogels have already been formulated to address this problem. For example, drug-loaded hydrogels such as mitomycin-C cross-linked HA hydrogels, were successfully tested for adhesion prevention. Seprafilm®, a carbodiimide-modified HA/carboxymethyl cellulose-based material, has been clinically tested and proven to be successful in reducing adhesion formations after gynecological procedures. Carbylan™-SX (PEGDA crosslinked CMHA-S hydrogel) has been shown to be effective in vocal fold repair by preventing scarring and ECM-based dysphonias. In addition, this composite was used for post-operative intra-abdominal and abdominopelvic adhesions preventions. HA haloacetate-based materials could further be used to improve the performance of currently available anti-adhesive biomaterials.

Medical device coating is another field that could benefit from the use of HA haloacetate-type biomaterials. Adsorption, ionic coupling, cross-linking, photochemical immobilization, covalent linking or biospecific immobilization are common procedures used for HA coating of medical devices. For example, endoluminal metallic stents, used for percutaneous coronary interventions, are commonly coated with biocompatible materials, because of the significant incidence of in-stent restenosis in patients that received non-coated stents (20% to 40% at 6 month after surgical intervention). Carbon, silicon carbide, gold or phosphorylcholine coated stents were previously used for neointimal hyperplasia prevention. Drug-coated stents that contained heparin (antithrombotic), dexamethasone (anti-inflammatory) or paclitaxel (anti-proliferative) were also developed. These coated materials were engineered to prevent or reduce thrombosis, inflammatory response and aberrant cell adhesion and proliferation. Although promising, many of the coating materials induced neointimal hyperplasia leading to restenosis and excessive inflammatory responses several months or even years after the surgical intervention.

The use of HA haloacetate-based coating materials could provide the awaited solution for restenosis prevention. Unmodified HA was already shown to be adherent to numerous scaffolds, and thus we suggest that HA haloacetates could be just as easily immobilized on commonly-used surgical scaffolds. In addition, the composition of HA haloacetate-based biomaterials would permit the modulation of post-surgical fibrotic responses. Their “living” structure would further allow for these materials to be chemically altered and tailored in an application-specific manner.

I. Crosslinkers and Preparation Thereof

Described herein are electrophilic crosslinkers. In one aspect, the crosslinker comprises the formula I 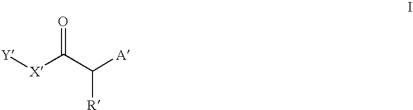
wherein
Y′ is a residue of a macromolecule selected from the group consisting of oligonucleotide, a nucleic acid or a metabolically stabilized analogue thereof, a polypeptide, a glycoprotein, a glycolipid, a polysaccharide and a protein;
   X′ is —O—, —S—, —NH—, or —NR″—;
   R′ is hydrogen, alkyl, perfluoroalkyl, aryl, heteroaryl, or halogen;
   R″ is hydrogen or C₁₋₅ alkyl; and
   A′ is a leaving group.

The macromolecule is any compound having at least one nucleophilic group that can displace a leaving group and form a new covalent bond. Examples of nucleophilic groups include, but are not limited to, hydroxyl, thiol, and substituted or unsubstituted groups. Referring to formula I, X′ is —O—, —S—, —NH—, or —NR″—. In another aspect, X′ is —O— or —NH—.

In another aspect, X′ is a residue of a nucleophilic group. In the case when the nucleophilic groups is a hydroxyl or amino groups, the hydroxyl or amino group is a free hydroxyl or amino group or it is derived from a carboxylic acid or amide, respectively. In one aspect, the macromolecule is an oligonucleotide, a nucleic acid or a metabolically stabilized analogue thereof, a polypeptide, a glycoprotein, or a glycolipid. In another aspect, the macromolecule is a polysaccharide or a protein. In another aspect, the macromolecule is a synthetic polymer. As used herein, metabolically stabilized analog refers to an analog in which a specific functional group that is labile to enzymatic or non-enzymatic degradation is altered by chemical modification to a different functional group that is more stable in vivo and in vitro, thereby extending the biological half-live of the analog.

Polysaccharides useful in the methods described herein have at least one nucleophilic group such as, for example, a hydroxyl group. In one aspect, the polysaccharide is a glycosaminoglycan (GAG). Glycosaminoglycans can be sulfated or non-sulfated. A GAG is one molecule with many alternating subunits. For example, HA is (GlcNAc-GlcUA-)x. Other GAGs are sulfated at different sugars. Generically, GAGs are represented by the formula A-B-A-B-A-B, where A is an uronic acid and B is an aminosugar that is either O- or N-sulfated, where the A and B units can be heterogeneous with respect to epimeric content or sulfation. Any natural or synthetic polymer containing uronic acid can be used. In one aspect, Y′ in formula I is a sulfated-GAG.

There are many different types of GAGs, having commonly understood structures, which, for example, are within the disclosed compositions, such as chondroitin sulfate, dermatan, heparan, heparin, dermatan sulfate, and heparan sulfate. Any GAG known in the art can be used in any of the methods described herein. Alginic acid, pectin, chitosan, and carboxymethylcellulose are among other polysaccharides useful in the methods described herein.

In another aspect, the polysaccharide Y′ in formula I is hyaluronan (HA). HA is a non-sulfated GAG. Hyaluronan is a well-known, naturally occurring, water soluble polysaccharide composed of two alternatively linked sugars, D-glucuronic acid and N-acetylglucosamine. The polymer is hydrophilic and highly viscous in aqueous solution at relatively low solute concentrations. It often occurs naturally as the sodium salt, sodium hyaluronate. Methods of preparing commercially available hyaluronan and salts thereof are well known. Hyaluronan can be purchased from Seikagaku Company, Novozymes Biopolymer, Novomatrix, Pharmacia Inc., Sigma Inc., and many other suppliers. For high molecular weight hyaluronan it is often in the range of 100 to 10,000 disaccharide units. In another aspect, the lower limit of the molecular weight of the hyaluronan is from 10,000, 20,000, 30,000, 40,000, 50,000, 60,000, 70,000, 80,000, 90,000, or 100,000, and the upper limit is 200,000, 300,000, 400,000, 500,000, 600,000, 700,000, 800,000, 900,000, or 1,000,000, where any of the lower limits can be combined with any of the upper limits.

In one aspect, Y′ in formula I can also be a synthetic polymer. The synthetic polymer has at least one nucleophilic group. In one aspect, the synthetic polymer residue in formula I comprises polyvinyl alcohol, polyethyleneimine, polyethylene glycol, polypropylene glycol, a polyol, a polyamine, a triblock polymer of polypropylene oxide-polyethylene oxide-polypropylene oxide, a star polymer of polyethylene glycol, or a dendrimer of polyethylene glycol.

In another aspect, Y′ in formula I is a protein. Proteins useful herein include, but are not limited to, an extracellular matrix protein, a chemically-modified extracellular matrix protein, or a partially hydrolyzed derivative of an extracellular matrix protein. The proteins may be naturally occurring or recombinant polypeptides possessing a cell interactive domain. The protein can also be a mixture of proteins, where one or more of the proteins are modified. Specific examples of proteins include, but are not limited to, collagen, elastin, decorin, laminin or fibronectin.

R′ in formula I comprises hydrogen, an alkyl group, a perfluoroalkyl group, an aryl group, a heteroaryl group or a halogen. In one aspect, R′ is hydrogen. In another aspect, R′ is a methyl group.

A′ in formula I comprises a leaving group. A leaving group is any group that can be displaced by a nucleophile. Several leaving groups are known in the art. Examples include, but are not limited to, halogens, alkoxides, activated esters, and the like. In one aspect, A′ in formula I is chloride, bromide, or iodide.

In one aspect, Y′ comprises a residue of an N-acetyl-glucosamine, wherein at least one primary C-6 hydroxyl group of the N-acetyl-glucosamine residue is substituted with (or attached to) the group —C(O)CH(R)(A′). In another aspect, Y′ comprises a residue of a N-acetyl-glucosamine, wherein at least one primary C-6 hydroxyl group of the N-acetyl-glucosamine residue is substituted with the group —C(O)CH(R)(A′), and at least one secondary hydroxyl group is substituted with the group —C(O)CH(R′)(A′). In a further aspect, Y′ comprises a residue of a N-acetyl-glucosamine, wherein at least one primary C-6 hydroxyl group of the N-acetyl-glucosamine residue is substituted with the group —C(O)CH(R′)(A′), and wherein from one primary C-6 hydroxyl group of the N-acetyl-glucosamine residue to about 100%, or substantially all, of the primary C-6 hydroxyl groups of the N-acetyl-glucosamine residue are substituted with the group —C(O)CH(R′)(A′). In another aspect, Y′ is a residue of a hyaluronan, wherein at least one hydroxyl group is substituted with —C(O)CH₂Cl, —C(O)CH₂Br, or —C(O)CH₂I.

Described herein are methods for making compounds having the formula I. In one aspect, the method comprises reacting a macromolecule comprising at least one nucleophilic group with a compound comprising the formula XV 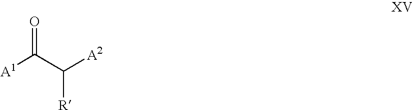
wherein

R′ comprises hydrogen or an alkyl group; and

A¹ and A² comprise the same or different leaving group.

The compounds having the formula XV cover a number of different molecules that can react with a macromolecule. Examples include, but are not limited to, activated esters, acyl halides, anhydrides, and the like.

In one aspect, R′ in formula XV is hydrogen. In another aspect, A¹ in formula XV forms a compound of the formula XVI 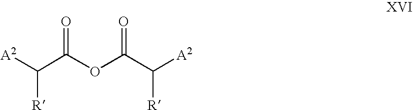
wherein

R′ comprises hydrogen or an alkyl group, wherein both R′ are the same group; and A² comprises the same leaving group as above.

Formula XVI covers symmetrical anhydrides; however, as discussed above, mixed anhydrides (e.g., where R′ and/or A² are not the same) are contemplated. In one aspect, R′ in formula XVI is hydrogen. In another aspect, A² in formula XVI comprises a halogen (e.g., chloride, bromide, or iodide). In a further aspect, the compound comprising formula XV is chloroacetic anhydride, bromoacetic anhydride, or iodoacetic anhydride.

Any of the macromolecules described herein can be reacted with the compound having the formula XV to produce an electrophilic macromolecule. In certain aspects, the nucleophilic group present on the macromolecule is a hydroxyl group or a substituted or unsubstituted amino group. In one aspect, the macromolecule comprises a glycosaminoglycan such as, for example, hyaluronan. In another aspect, the macromolecule is hyaluronan and the compound having the formula XV is chloroacetic anhydride, bromoacetic anhydride, or iodoacetic anhydride.

The reaction between the macromolecule and the compound having the formula XV can be conducted at various reaction temperatures and times, which will vary depending upon the selection of starting materials. The selection of solvents will also vary on the solubility of the starting materials. In certain aspects, it is desirable to conduct the reaction at a pH greater than 7. For example, when the macromolecule has one or more hydroxyl groups, a basic medium may be desired to deprotonate a certain number of the hydroxyl groups and facilitate the reaction between the macromolecule and the compound having the formula XV.

Any of the compounds described herein can be the pharmaceutically-acceptable salt or ester thereof. In one aspect, pharmaceutically-acceptable salts are prepared by treating the free acid with an appropriate amount of a pharmaceutically-acceptable base. Representative pharmaceutically-acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like. In one aspect, the reaction is conducted in water, alone or in combination with an inert, water-miscible organic solvent, at a temperature of from about 0° C. to about 100° C. such as at room temperature. In certain aspects where applicable, the molar ratio of the compounds described herein to base used are chosen to provide the ratio desired for any particular salts. For preparing, for example, the ammonium salts of the free acid starting material, the starting material can be treated with approximately one equivalent of pharmaceutically-acceptable base to yield a neutral salt.

In another aspect, if the compound possesses a basic group, it can be protonated with an acid such as, for example, HCl, HBr, or H₂SO₄, to produce the cationic salt. In one aspect, the reaction of the compound with the acid or base is conducted in water, alone or in combination with an inert, water-miscible organic solvent, at a temperature of from about 0° C. to about 100° C. such as at room temperature. In certain aspects where applicable, the molar ratio of the compounds described herein to base used are chosen to provide the ratio desired for any particular salts. For preparing, for example, the ammonium salts of the free acid starting material, the starting material can be treated with approximately one equivalent of pharmaceutically-acceptable base to yield a neutral salt.

Ester derivatives are typically prepared as precursors to the acid form of the compounds. Generally, these derivatives will be lower alkyl esters such as methyl, ethyl, and the like. Amide derivatives —(CO)NH₂, —(CO)NHR and —(CO)NR₂, where R is an alkyl group defined above, can be prepared by reaction of the carboxylic acid-containing compound with ammonia or a substituted amine.

II. Coupling of Macromolecules

The compounds having the formula I are electrophilic, and can react with one or more macromolecules possessing nucleophilic groups to couple the macromolecules. In one aspect, a method for coupling two or more macromolecules comprises reacting a first macromolecule comprising the formula I with a second macromolecule comprising at least one nucleophilic group. It is contemplated that the first macromolecule comprising formula I can have a plurality of electrophilic groups and the second macromolecule can have a plurality of nucleophilic groups. Thus, it is possible to produce a matrix or network of different macromolecules.

In one aspect, the second macromolecule has the formula II 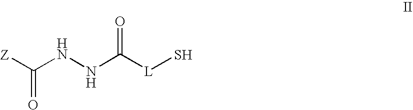
wherein
Z is a residue of a macromolecule, and
L is a polyalkylene group, a polyether group, a polyamide group, a polyamino group, an aryl group, a polyester, or a polythioether group.

The macromolecule residue Z can be any of the macromolecules described above. In one aspect, the second macromolecule can be a protein having at least one thiol group. In this aspect, the protein can be naturally occurring or synthetic. In one aspect, the protein comprises an extracellular matrix protein or a chemically-modified extracellular matrix protein. In another aspect, the protein comprises collagen, elastin, decorin, laminin, or fibronectin. In one aspect, the protein comprises genetically engineered proteins with additional thiol groups (e.g., cysteine residues). In a further aspect, the protein comprises a synthetic polypeptide that can be a branched (e.g., a dendrimer) or linear with additional thiol groups (e.g., cysteine residues).

In another aspect, L in formula II is a polyalkylene group. In another aspect, L in formula II is a —CH₂— or a C₂ to C₂₀ polyalkylene group. In another aspect, L in formula II is CH₂CH₂ or CH₂CH₂CH₂. In one aspect, Z is a residue of hyaluronan and L in formula II is CH₂CH₂ or CH₂CH₂CH₂. In a further aspect, Z is a residue of gelatin and L in formula II is CH₂CH₂ or CH₂CH₂CH₂. In one aspect, L in formula II is, independently, CH₂CH₂ or CH₂CH₂CH₂. In another aspect, Z is a residue of hyaluronan.

In one aspect, the second macromolecule comprises the formula XX
Y—X—R—SH XX
wherein

Y is a residue of a macromolecule;

X is O, NH or a residue of a nucleophilic group; and

R comprises a substituted or unsubstituted C₂ or C₃ alkyl group.

In one aspect, X is O or NH, or where X is a residue of a hydroxyl group or an amino group.

The macromolecule Y in formula XX can be any of the macromolecules described herein. In one aspect, the macromolecule comprises an oligonucleotide, a nucleic acid or a metabolically stabilized analogue thereof, a polypeptide, a glycoprotein, a glycolipid, or a pharmaceutically-acceptable compound. In one aspect, Y comprises a residue of a glycosaminoglycan. In another aspect, Y comprises a residue of hyaluronan. In a further aspect, Y comprises a residue of an N-acetyl-glucosamine, wherein at least one primary C-6 hydroxyl group of the N-acetyl-glucosamine residue is substituted with the group —RSH. Further to this aspect, at least one secondary hydroxyl group is substituted with the group —RSH as well. In another aspect, one primary C-6 hydroxyl group of the N-acetyl-glucosamine residue to 100% of the primary C-6 hydroxyl groups of the N-acetyl-glucosamine residue are substituted with the group —RSH.

In another aspect, R in formula XX is CH₂CH₂, CH₂CH₂CH₂, CH₂CHR⁵, CHR⁵CHR⁵, C(R⁵)₂CHR⁵, or C(R⁵)₂C(R⁵)₂, wherein R⁵ is an alkyl group. In one aspect, Y in formula XX is a residue of a hyaluronan, wherein at least one hydroxyl group is substituted with —CH₂CH₂SH.

The second macromolecule having the formula XX can be synthesized by the methods described herein. In one aspect, the method comprises reacting a macromolecule comprising at least one nucleophilic group (e.g., hydroxyl group or amino group) with a compound comprising the formula XVII 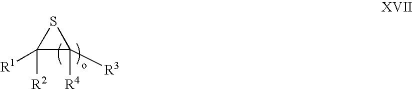
wherein R¹, R², R³, and R⁴ are, independently, hydrogen, an alkyl group, a perfluoroalkyl group, an aryl group, or a heteroaryl group, and o is 1 or 2.

In one aspect, o in formula XVII is 1. In another aspect, o in formula XVII is 1 and R¹-R⁴ are hydrogen. In another aspect, the second macromolecule comprises the reaction product between hyaluronan and a compound having the formula XVII, where o is 1 and R¹-R⁴ are hydrogen.

The reaction between the macromolecule and the compound having the formula XV can be conducted at various reaction temperatures and times, which will vary depending upon the selection of starting materials. The selection of solvents will also vary on the solubility of the starting materials. In certain aspects, it is desirable to conduct the reaction at a pH greater than 7. For example, when the macromolecule has one or more hydroxyl groups, a basic medium may be desired to deprotonate a certain number of the hydroxyl groups and facilitate the reaction between the macromolecule and the compound having the formula XVII.

In one aspect, the coupling of the first and second macromolecules can be conducted at a pH of from 7 to 12, 7.5 to 11, 7.5 to 10, or 7.5 to 9.5, or a pH of 8. In one aspect, the solvent used can be water (alone) or an aqueous containing organic solvent. In one aspect, when the mixed solvent system is used, a base such as a primary, secondary, or tertiary amine can be used. In one aspect, an excess of first macromolecule having the formula I is used relative to the second macromolecule in order to ensure that all of the second macromolecule is consumed during the reaction. Depending upon the selection of the first and second macromolecule, the pH of the reaction, and the solvent selected, coupling can occur from within minutes to several days.

The compounds described herein have at least one fragment comprising the formula VII 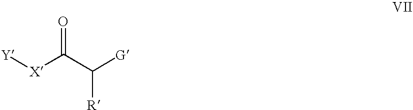
wherein

Y′ is a residue of a first macromolecule;

X′ is —O—, —S, —S—, —NH—, or —NR″—;

R″ is hydrogen or C₁₋₅ alkyl;

R′ comprises hydrogen or an alkyl group; and

G′ comprises a residue of a second macromolecule.

The term “fragment” as used herein refers to the entire molecule itself or a portion or segment of a larger molecule. For example, Y′ in formula VII may be a high molecular weight polysaccharide that is crosslinked with another polysaccharide, synthetic polymer, or thiolated polymer to produce the coupled compound. The compound has at a minimum one unit depicted in formula VII, which represents the reaction product between at least one first macromolecule and a second macromolecule.

The compounds having the formula I possess electrophilic groups that have numerous advantages when compared to other macromolecules with acrylate groups, which are also electrophilic. For example, acrylate groups are photoreactive and can react with other macromolecules possessing acrylate groups. The compounds having the formula I do not react with each other and are free to react with other macromolecules (e.g., thiolated macromolecules). Additionally, due to the leaving group present in formula I, the compounds are generally hydrolyzable. This is particularly desirable in physiological conditions, where the compound having the formula I can be hydrolyzed by the subject over time to produce a compound that is less toxic or not toxic at all.

III. Pharmaceutical Compositions

In one aspect, any of the compounds produced by the methods described above can further include at least one pharmaceutically-acceptable compound (or biologically active agent). The resulting pharmaceutical composition can provide a system for sustained, continuous delivery of drugs and other biologically-active agents to tissues adjacent to or distant from the application site. The biologically-active agent is capable of providing a local or systemic biological, physiological or therapeutic effect in the biological system to which it is applied. For example, the agent can act to control infection or inflammation, enhance cell growth and tissue regeneration, control tumor growth, act as an analgesic, promote anti-cell attachment, and enhance bone growth, among other functions. Additionally, any of the compounds described herein can contain combinations of two or more pharmaceutically-acceptable compounds.

In one aspect, the pharmaceutically-acceptable compounds can include substances capable of preventing an infection systemically in the biological system or locally at the defect site, as for example, anti-inflammatory agents such as, but not limited to, pilocarpine, hydrocortisone, prednisolone, cortisone, diclofenac sodium, indomethacin, 6∝-methyl-prednisolone, corticosterone, dexamethasone, prednisone, and the like; antibacterial agents including, but not limited to, penicillin, cephalosporins, bacitracin, tetracycline, doxycycline, gentamycin, chloroquine, vidarabine, and the like; analgesic agents including, but not limited to, salicylic acid, acetaminophen, ibuprofen, naproxen, piroxicam, flurbiprofen, morphine, and the like; local anesthetics including, but not limited to, cocaine, lidocaine, benzocaine, and the like; immunogens (vaccines) for stimulating antibodies against hepatitis, influenza, measles, rubella, tetanus, polio, rabies, and the like; peptides including, but not limited to, leuprolide acetate (an LH-RH agonist), nafarelin, and the like. All compounds are commercially available.

In one aspect, the pharmaceutically-acceptable compound can be a growth factor. Any substance or metabolic precursor that is capable of promoting growth and survival of cells and tissues or augmenting the functioning of cells is useful as a growth factor. Examples of growth factors include, but are not limited to, a nerve growth promoting substance such as a ganglioside, a nerve growth factor, and the like; a hard or soft tissue growth promoting agent such as fibronectin (FN), human growth hormone (HGH), a colony stimulating factor, bone morphogenic protein, platelet-derived growth factor (PDGF), insulin-derived growth factor (IGF-I, IGF-II), transforming growth factor-alpha (TGF-alpha), transforming growth factor-beta (TGF-beta), epidermal growth factor (EGF), fibroblast growth factor (FGF), interleukin-1 (IL-1), vascular endothelial growth factor (VEGF) and keratinocyte growth factor (KGF), bone-derived bone material (e.g., demineralized bone matrix), and the like; and antineoplastic agents such as methotrexate, 5-fluorouracil, adriamycin, vinblastine, cisplatin, tumor-specific antibodies conjugated to toxins, tumor necrosis factor, and the like.

Any of the growth factors disclosed in U.S. Pat. No. 6,534,591 B2, which is incorporated by reference in its entirety, can be used in this aspect. In one aspect, the growth factor includes transforming growth factors (TGFs), fibroblast growth factors (FGFs), platelet derived growth factors (PDGFs), epidermal growth factors (EGFs), connective tissue activated peptides (CTAPs), osteogenic factors, and biologically active analogs, fragments, and derivatives of such growth factors. Members of the transforming growth factor (TGF) supergene family, which are multifunctional regulatory proteins. Members of the TGF supergene family include the beta transforming growth factors (for example, TGF-β1, TGF-β2, TGF-β3); bone morphogenetic proteins (for example, BMP-1, BMP-2, BMP-3, BMP-4, BMP-5, BMP-6, BMP-7, BMP-8, BMP-9); heparin-binding growth factors (for example, fibroblast growth factor (FGF), epidermal growth factor (EGF), platelet-derived growth factor (PDGF), insulin-like growth factor (IGF)); inhibins (for example, Inhibin A, Inhibin B); growth differentiating factors (for example, GDF-1); and Activins (for example, Activin A, Activin B, Activin AB).

Growth factors can be isolated from native or natural sources, such as from mammalian cells, or can be prepared synthetically, such as by recombinant DNA techniques or by various chemical processes. In addition, analogs, fragments, or derivatives of these factors can be used, provided that they exhibit at least some of the biological activity of the native molecule. For example, analogs can be prepared by expression of genes altered by site-specific mutagenesis or other genetic engineering techniques.

Other useful substances include hormones such as progesterone, testosterone, and follicle stimulating hormone (FSH) (birth control, fertility-enhancement), insulin, and the like; antihistamines such as diphenhydramine, and the like; cardiovascular agents such as papaverine, streptokinase and the like; anti-ulcer agents such as isopropamide iodide, and the like; bronchodilators such as metaprotemal sulfate, aminophylline, and the like; vasodilators such as theophylline, niacin, minoxidil, and the like; central nervous system agents such as tranquilizer, B-adrenergic blocking agent, dopamine, and the like; antipsychotic agents such as risperidone, narcotic antagonists such as naltrexone, naloxone, buprenorphine; and other like substances. All compounds are commercially available.

The pharmaceutical compositions can be prepared using techniques known in the art. In one aspect, the composition is prepared by admixing a compound described herein with a pharmaceutically-acceptable compound. The term “admixing” is defined as mixing the two components together so that there is no chemical reaction or physical interaction. The term “admixing” also includes the chemical reaction or physical interaction between the compound and the pharmaceutically-acceptable compound. Covalent bonding to reactive therapeutic drugs, e.g., those having nucleophilic groups, can be undertaken on the compound. Second, non-covalent entrapment of a pharmacologically active agent in a cross-linked polysaccharide is also possible. Third, electrostatic or hydrophobic interactions can facilitate retention of a pharmaceutically-acceptable compound in a modified polysaccharide.

It will be appreciated that the actual preferred amounts of active compound in a specified case will vary according to the specific compound being utilized, the particular compositions formulated, the mode of application, and the particular situs and subject being treated. Dosages for a given host can be determined using conventional considerations, e.g. by customary comparison of the differential activities of the subject compounds and of a known agent, e.g., by means of an appropriate conventional pharmacological protocol. Physicians and formulators, skilled in the art of determining doses of pharmaceutical compounds, will have no problems determining dose according to standard recommendations (Physicians Desk Reference, Barnhart Publishing (1999).

Pharmaceutical compositions described herein can be formulated in any excipient the biological system or entity can tolerate. Examples of such excipients include, but are not limited to, water, saline, Ringer's solution, dextrose solution, Hank's solution, and other aqueous physiologically balanced salt solutions. Nonaqueous vehicles, such as fixed oils, vegetable oils such as olive oil and sesame oil, triglycerides, propylene glycol, polyethylene glycol, and injectable organic esters such as ethyl oleate can also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal, cresols, formalin and benzyl alcohol.

Pharmaceutical carriers are known to those skilled in the art. These most typically would be standard carriers for administration to humans, including solutions such as sterile water, saline, and buffered solutions at physiological pH.

Molecules intended for pharmaceutical delivery can be formulated in a pharmaceutical composition. Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally).

Preparations for administration include sterile aqueous or non-aqueous solutions, suspensions, and emulsions. Examples of non-aqueous carriers include water, alcoholic/aqueous solutions, emulsions or suspensions, including saline and buffered media. Parenteral vehicles, if needed for collateral use of the disclosed compositions and methods, include sodium chloride solution, Ringer's dextrose, dextrose and sodium chloride, lactated Ringer's, or fixed oils. Intravenous vehicles, if needed for collateral use of the disclosed compositions and methods, include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Dosing is dependent on severity and responsiveness of the condition to be treated, but will normally be one or more doses per day, with course of treatment lasting from several days to several months or until one of ordinary skill in the art determines the delivery should cease. Persons of ordinary skill can easily determine optimum dosages, dosing methodologies and repetition rates.

In one aspect, any of the compounds and pharmaceutical compositions can include living cells. Examples of living cells include, but are not limited to, stem cells, fibroblasts, hepatocytes, chondrocytes, stem cells, bone marrow, muscle cells, cardiac myocytes, neuronal cells, or pancreatic islet cells.

IV. Methods of Use

The compounds and pharmaceutical compositions described herein (e.g., compounds having the formula I and coupled macromolecules derived from compounds having the formula I) can be used for a variety of uses related to drug delivery, small molecule delivery, wound healing, burn injury healing, and tissue regeneration/engineering. The disclosed compositions are useful for situations that benefit from a hydrated, pericellular environment in which assembly of other matrix components, presentation of growth and differentiation factors, cell migration, or tissue regeneration are desirable.

The compounds and compositions described herein can improve wound healing in a subject in need of such improvement, comprising contacting the wound of the subject with one or more compounds of claims. The compounds and pharmaceutical compositions described herein can be placed directly in or on any biological system without purification as it is composed of biocompatible materials. Examples of sites the compounds can be placed include, but not limited to, soft tissue such as muscle or fat; hard tissue such as bone or cartilage; areas of tissue regeneration; a void space such as periodontal pocket; surgical incision or other formed pocket or cavity; a natural cavity such as the oral, vaginal, rectal or nasal cavities, the cul-de-sac of the eye, and the like; the peritoneal cavity and organs contained within, and other sites into or onto which the compounds can be placed including a skin surface defect such as a cut, scrape or burn area. It is contemplated that the tissue can be damaged due to injury or a degenerative condition or, in the alternative, the compounds and compositions described herein can be applied to undamaged tissue to prevent injury to the tissue. The present compounds can be biodegradable and naturally occurring enzymes will act to degrade them over time. Components of the compound can be “bioabsorbable” in that the components of the compound will be broken down and absorbed within the biological system, for example, by a cell, tissue and the like. Additionally, the compounds, especially compounds that have not been rehydrated, can be applied to a biological system to absorb fluid from an area of interest.

The compounds and compositions described herein can deliver at least one pharmaceutically-acceptable compound to a patient in need of such delivery, comprising contacting at least one tissue capable of receiving the pharmaceutically-acceptable compound with one or more compositions described herein. The compounds described herein can be used as a carrier for a wide variety of releasable biologically active substances having curative or therapeutic value for human or non-human animals. Many of these substances that can be carried by the compound are discussed above. Included among biologically active materials which are suitable for incorporation into the gels of the invention are therapeutic drugs, e.g., anti-inflammatory agents, anti-pyretic agents, steroidal and non-steroidal drugs for anti-inflammatory use, hormones, growth factors, contraceptive agents, antivirals, antibacterials, antifungals, analgesics, hypnotics, sedatives, tranquilizers, anti-convulsants, muscle relaxants, local anesthetics, antispasmodics, antiulcer drugs, peptidic agonists, sympathomimetic agents, cardiovascular agents, antitumor agents, oligonucleotides and their analogues and so forth. A biologically active substance is added in pharmaceutically active amounts.

In one aspect, the compounds and compositions described herein can be used for the delivery of living cells to a subject. Any of the living cells described herein can be used in the aspect.

In one aspect, the compounds and compositions can be used for the delivery of growth factors and molecules related to growth factors. For example the growth factors can be a nerve growth promoting substance such as a ganglioside, a nerve growth factor, and the like; a hard or soft tissue growth promoting agent such as fibronectin (FN), human growth hormone (HGH), a colony stimulating factor, bone morphogenic protein, platelet-derived growth factor (PDGF), insulin-derived growth factor (IGF-I, IGF-II), transforming growth factor-alpha (TGF-alpha), transforming growth factor-beta (TGF-beta), epidermal growth factor (EGF), fibroblast growth factor (FGF), interleukin-1 (IL-1). Preferred growth factors are bFGF and TGF-β. Also preferred are vascular endothelial growth factor (VEGF) and keratinocyte growth factor (KGF).

In another aspect, anti-inflammatories such as ibuprofen, naproxen, ketoprofen and indomethacin can be used. Other biologically active substances are peptides, which are naturally occurring, non-naturally occurring or synthetic polypeptides or their isosteres, such as small peptide hormones or hormone analogues and protease inhibitors. Spermicides, antibacterials, antivirals, antifungals and antiproliferatives such as fluorodeoxyuracil and adriamycin can also be used. These substances are all known in the art and commercially available.

The term “therapeutic drugs” as used herein is intended to include those defined in the Federal Food, Drug and Cosmetic Act. The United States Pharmacopeia (USP) and the National Formulary (NF) are the recognized standards for potency and purity for most common drug products.

In one aspect, the pharmaceutically acceptable compound is pilocarpine, hydrocortisone, prednisolone, cortisone, diclofenac sodium, indomethacin, 6%-methyl-prednisolone, corticosterone, dexamethasone and prednisone. However, methods are also provided wherein delivery of a pharmaceutically-acceptable compound is for a medical purpose. Examples of medical purposes include, but are not limited to, the delivery of contraceptive agents, treating postsurgical adhesions, promoting skin growth, preventing scarring, dressing wounds, conducting viscosurgery, conducting viscosupplementation, and engineering tissue.

The rate of drug delivery depends on the hydrophobicity of the molecule being released. Hydrophobic molecules, such as dexamethasone and prednisone are released slowly from the compound as it swells in an aqueous environment, while hydrophilic molecules, such as pilocarpine, hydrocortisone, prednisolone, cortisone, diclofenac sodium, indomethacin, 6∝-methyl-prednisolone and corticosterone, are released quickly. The ability of the compound to maintain a slow, sustained release of steroidal anti-inflammatories makes the compounds described herein extremely useful for wound healing after trauma or surgical intervention. Additionally, the compound can be used as a barrier system for enhancing cell growth and tissue regeneration.

In certain methods the delivery of molecules or reagents related to angiogenesis and vascularization are achieved. Disclosed are methods for delivering agents, such as VEGF, that stimulate microvascularization. Also disclosed are methods for the delivery of agents that can inhibit angiogenesis and vascularization, such as those compounds and reagents useful for this purpose disclosed in but not limited to U.S. Pat. Nos. 6,174,861 for “Methods of inhibiting angiogenesis via increasing in vivo concentrations of endostatin protein;” 6,086,865 for “Methods of treating angiogenesis-induced diseases and pharmaceutical compositions thereof;” 6,024,688 for “Angiostatin fragments and method of use;” 6,017,954 for “Method of treating tumors using O-substituted fumagillol derivatives;” 5,945,403 for “Angiostatin fragments and method of use;” 5,892,069 “Estrogenic compounds as anti-mitotic agents;” for 5,885,795 for “Methods of expressing angiostatic protein;” 5,861,372 for “Aggregate angiostatin and method of use;” 5,854,221 for “Endothelial cell proliferation inhibitor and method of use;” 5,854,205 for “Therapeutic antiangiogenic compositions and methods;” 5,837,682 for “Angiostatin fragments and method of use;” 5,792,845 for “Nucleotides encoding angiostatin protein and method of use;” 5,733,876 for “Method of inhibiting angiogenesis;” 5,698,586 for “Angiogenesis inhibitory agent;” 5,661,143 for “Estrogenic compounds as anti-mitotic agents;” 5,639,725 for “Angiostatin protein;” 5,504,074 for “Estrogenic compounds as anti-angiogenic agents;” 5,290,807 for “Method for regressing angiogenesis using o-substituted fumagillol derivatives;” and 5,135,919 for “Method and a pharmaceutical composition for the inhibition of angiogenesis” which are herein incorporated by reference for the material related to molecules for angiogenesis inhibition.

Described herein are methods for improving wound healing in a subject in need of such improvement by contacting any of the compounds or pharmaceutical compositions described herein with a wound of a subject in need of wound healing improvement. Also provided are methods to deliver at least one pharmaceutically-acceptable compound to a patient in need of such delivery by contacting any of the compounds or pharmaceutical compositions described herein with at least one tissue capable of receiving said pharmaceutically-acceptable compound.

The disclosed compositions can be used for treating a wide variety of tissue defects in a subject, for example, a tissue with a void such as a periodontal pocket, a shallow or deep cutaneous wound, a surgical incision, a bone or cartilage defect, and the like. For example, the compounds described herein can be in the form of a hydrogel film. The hydrogel film can be applied to a defect in bone tissue such as a fracture in an arm or leg bone, a defect in a tooth, a cartilage defect in the joint, ear, nose, or throat, and the like. The hydrogel film composed of the compound described herein can also function as a barrier system for guided tissue regeneration by providing a surface on or through which the cells can grow. To enhance regeneration of a hard tissue such as bone tissue, it is preferred that the hydrogel film provides support for new cell growth that will replace the matrix as it becomes gradually absorbed or eroded by body fluids.

The hydrogel film composed of a compound described herein can be delivered onto cells, tissues, and/or organs, for example, by injection, spraying, squirting, brushing, painting, coating, and the like. Delivery can also be via a cannula, catheter, syringe with or without a needle, pressure applicator, pump, and the like. The compound can be applied onto a tissue in the form of a film, for example, to provide a film dressing on the surface of the tissue, and/or to adhere to a tissue to another tissue or hydrogel film, among other applications.

In one aspect, the compounds described herein are administered via injection. For many clinical uses, when the compound is in the form of a hydrogel film, injectable hydrogels are preferred for three main reasons. First, an injectable hydrogel could be formed into any desired shape at the site of injury. Because the initial hydrogels can be sols or moldable putties, the systems can be positioned in complex shapes and then subsequently crosslinked to conform to the required dimensions. Second, the hydrogel would adhere to the tissue during gel formation, and the resulting mechanical interlocking arising from surface microroughness would strengthen the tissue-hydrogel interface. Third, introduction of an in situ-crosslinkable hydrogel could be accomplished using needle or by laparoscopic methods, thereby minimizing the invasiveness of the surgical technique.

The compounds described herein can be used to treat periodontal disease, gingival tissue overlying the root of the tooth can be excised to form an envelope or pocket, and the composition delivered into the pocket and against the exposed root. The compounds can also be delivered to a tooth defect by making an incision through the gingival tissue to expose the root, and then applying the material through the incision onto the root surface by placing, brushing, squirting, or other means.

When used to treat a defect on skin or other tissue, the compounds described herein can be in the form of a hydrogel film that can be placed on top of the desired area. In this aspect, the hydrogel film is malleable and can be manipulated to conform to the contours of the tissue defect.

It is understood that the disclosed compositions and methods can be applied to a subject in need of tissue regeneration. For example, cells can be incorporated into the compounds described herein for implantation. In one aspect the subject is a mammal. Preferred mammals to which the compositions and methods apply are mice, rats, cows or cattle, horses, sheep, goats, cats, dogs, ferrets, and primates, including apes, chimpanzees, orangutans, and humans. In another aspect, the compounds and compositions described herein can be applied to birds.

When being used in areas related to tissue regeneration such as wound or burn healing, it is not necessary that the disclosed methods and compositions eliminate the need for one or more related accepted therapies. It is understood that any decrease in the length of time for recovery or increase in the quality of the recovery obtained by the recipient of the disclosed compositions or methods has obtained some benefit. It is also understood that some of the disclosed compositions and methods can be used to prevent or reduce fibrotic adhesions occurring as a result of wound closure as a result of trauma, such surgery. It is also understood that collateral affects provided by the disclosed compositions and compounds are desirable but not required, such as improved bacterial resistance or reduced pain etc.

In one aspect, the compounds described herein can be used to repair a damaged elastic tissue in a subject, comprising contacting the damaged tissue with one or more compounds described herein. The source of the damaged tissue can be due to an injury or by a degenerative condition. Examples of elastic tissues include, but are not limited to, a vocal cord, a cardiovascular tissue, a muscle, a tendon, a ligament, bladder tissue, tissue in the urethra, a sphincter muscle, or a muscle in the gastrointestinal tract.

The compounds described herein can be used as substrates for growing and differentiating cells. For example, the compounds and compositions described herein can be formed into a laminate, a gel, a bead, a sponge, a film, a mesh, an electrospun nanofiber, a woven mesh, or a non-woven mesh. In one aspect, described herein is a method for growing a plurality of cells, comprising (a) depositing a parent set of cells on a substrate described herein, and (b) culturing the substrate with the deposited cells to promote the growth of the cells.

In another aspect, described herein is a method for differentiating cells, comprising (a) depositing a parent set of cells on a substrate described herein, and (b) culturing the assembly to promote differentiation of the cells.

Many types of cells can be grown and/or differentiated using the substrates described herein including, but not limited to, stem cells, committed stem cells, differentiated cells, and tumor cells. Examples of stem cells include, but are not limited to, embryonic stem cells, bone marrow stem cells and umbilical cord stem cells. Other examples of cells used in various embodiments include, but are not limited to, osteoblasts, myoblasts, neuroblasts, fibroblasts, glioblasts, germ cells, hepatocytes, chondrocytes, epithelial cells, cardiovascular cells, keratinocytes, smooth muscle cells, cardiac muscle cells, connective tissue cells, glial cells, epithelial cells, endothelial cells, hormone-secreting cells, cells of the immune system, and neurons.

Cells useful herein can be cultured in vitro, derived from a natural source, genetically engineered, or produced by any other means. Any natural source of prokaryotic or eukaryotic cells can be used. It is also contemplated that cells can be cultured ex vivo.

Atypical or abnormal cells such as tumor cells can also be used herein. Tumor cells cultured on substrates described herein can provide more accurate representations of the native tumor environment in the body for the assessment of drug treatments. Growth of tumor cells on the substrates described herein can facilitate characterization of biochemical pathways and activities of the tumor, including gene expression, receptor expression, and polypeptide production, in an in vivo-like environment allowing for the development of drugs that specifically target the tumor.

Cells that have been genetically engineered can also be used herein. The engineering involves programming the cell to express one or more genes, repressing the expression of one or more genes, or both. Genetic engineering can involve, for example, adding or removing genetic material to or from a cell, altering existing genetic material, or both. Embodiments in which cells are transfected or otherwise engineered to express a gene can use transiently or permanently transfected genes, or both. Gene sequences may be full or partial length, cloned or naturally occurring.

In another aspect, described herein is method for growing tissue, comprising (a) depositing a parent set of cells that are a precursor to the tissue on a substrate described herein, and (b) culturing the substrate with the deposited cells to promote the growth of the tissue. It is also contemplated that viable cells can be deposited on the substrates described herein and cultured under conditions that promote tissue growth. Tissue grown (i.e., engineered) from any of the cells described above is contemplated with the substrates described herein. The supports described herein can support many different kinds of precursor cells, and the substrates can guide the development of new tissue. The production of tissues has numerous applications in wound healing. Tissue growth can be performed in vivo or ex vivo using the methods described herein.

The compounds described herein can be applied to an implantable device such as a suture, claps, prosthesis, catheter, metal screw, bone plate, pin, a bandage such as gauze, and the like, to enhance the compatibility and/or performance or function of an implantable device with a body tissue in an implant site. The compounds can be used to coat the implantable device. For example, the compounds could be used to coat the rough surface of an implantable device to enhance the compatibility of the device by providing a biocompatible smooth surface that reduces the occurrence of abrasions from the contact of rough edges with the adjacent tissue.

The compounds can also be used to enhance the performance or function of an implantable device. For example, when the compound is a hydrogel film, the hydrogel film can be applied to a gauze bandage to enhance its compatibility or adhesion with the tissue to which it is applied. The hydrogel film can also be applied around a device such as a catheter or colostomy that is inserted through an incision into the body to help secure the catheter/colosotomy in place and/or to fill the void between the device and tissue and form a tight seal to reduce bacterial infection and loss of body fluid.

It is also contemplated that the compounds described herein can be used as a bio-artificial material that can be used as an implantable device in a subject. In this aspect, the bio-artificial material can be molded into any desired shape. In one aspect, the bio-artificial material comprises the reaction product between one or more compounds having the formula I and a macromolecule comprising at least two thiol groups. In one aspect, the macromolecule comprises an elastin-like peptide with at least two thiol groups. In another aspect, one or more bio-artificial materials can be used to produce a prosthetic device. In one aspect, the device can be living prosthetic device, where the device promotes tissue growth. Depending upon the composition of the bio-artificial material, the device can be deformable to fit the specific needs of the subject.

It is understood that any given particular aspect of the disclosed compositions and methods can be easily compared to the specific examples and embodiments disclosed herein, including the non-polysaccharide based reagents discussed in the Examples. By performing such a comparison, the relative efficacy of each particular embodiment can be easily determined. Particularly preferred compositions and methods are disclosed in the Examples herein, and it is understood that these compositions and methods, while not necessarily limiting, can be performed with any of the compositions and methods disclosed herein.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

Materials and Methods

Materials and Analytical Instrumentation. High molecular weight hyaluronan (HA, MW=824 kDa) was from Contipro C Co, Czech Republic. Bromoacetic anhydride (BA), hyaluronidase type I—S from bovine testes (HAse, 451 U/mg solid) were from Sigma-Aldrich Chemical Co., Milwaukee, Wis. Phosphate buffered saline 10× (PBS), sodium hydroxide (NaOH), hydrochloric acid 12.1 N (HCl), sodium iodide (NaI), dibasic sodium phosphate, heptahydrate (Na₂PO₄.7H₂O) and SpectraPor dialysis tubing MWCO 10.000 were from Fisher Scientific, Hanover Park, Ill. SAMSA fluorescein (5-((2-(and -3)-S-acetylmercapto)succinoyl)amino)fluorescein) mixed isomers was purchased from Molecular Probes Inc., Eugene, Oreg. T31 human tracheal scar fibroblasts were a generous gift from Dr. S. L. Thibeault (Division of Otolaryngology—Head and Neck Surgery, Department of Surgery, University of Utah, Salt Lake City, Utah; Division of Otolaryngology—Head and Neck Surgery, Department of Surgery, University of Wisconsin, Madison, Wis.).

¹H-NMR spectral data were acquired using a Varian INOVA 400 at 400 MHz. UV/VIS spectra and measurements were performed on a Hewlett-Packard 8453 UV-visible spectrometer, Palo Alto, Calif. Gel permeation chromatography (GPC) analysis was obtained using the following components: Waters 486 tunable absorbance detector, Waters 410 differential refractometer, Waters 515 HPLC pump and Ultrahydrogel 1000 column (7.8×300 mm) (Waters Corp., Milford, Mass.). The mobile phase for GPC consisted of 0.2 M PBS buffer/methanol (80:20 volume ratio). HA standards used to calibrate the system were from Novozymes Biopolymers, Bågsvaerd, Denmark. An OPTI Max microplate reader (Molecular Devices, Sunnyvale, Calif.) was used to determine the 490 nm absorbance values for cell viability assays.

Synthesis of Bromoacetate Derivatized Hyaluronan (HABA). Hyaluronan (6.0 g) was dissolved in 600 mL distilled water (1% w/v solution). The pH of the solution was adjusted to 9.0 by adding 1 M NaOH. Bromoacetic anhydride (40 g, 153 mmol) was then added dropwise to the solution and the reaction was stirred for 24 h at 4° C. This amount of bromoacetic anhydride corresponds to 10 equivalents relative to the number of primary C-6 hydroxyl groups of the N-acetylglucosamine residues. The reaction mixture was then dialyzed (MWCO 10000) for 3 days against distilled water. The sample was then lyophilized and analyzed. The purity of the sample was determined by ¹H-NMR and GPC and the degree of substitution (SD) was determined derivatization with SAMSA fluorescein (SD˜18%). ¹H-NMR (D₂O); chemical shift corresponding to the substituent: δ=3.84 ppm (COCH₂Br).

Synthesis of Iodoacetate Derivatized Hyaluronan (HAIA). HABA (2.15 g) was dissolved in 215 mL distilled water (1% w/v solution) and reacted with 10 equivalents of NaI. The reaction was stirred overnight at room temperature. Next, the reaction mixture was dialyzed for 3 days (MWCO 10000) and subsequently lyophilized. The purity of the modified hyaluronan was determined by ¹H-NMR and GPC. The degree of substitution was calculated by SAMSA fluorescein derivatization (SD˜19%). ¹H-NMR (D₂O); chemical shift corresponding to the substituent: δ=3.7 ppm (COCH₂I).

SAMSA Fluorescein Derivatization. SAMSA fluorescein (25 mg) was dissolved in 2.5 mL of 0.1 M NaOH and incubated for 15 min at room temperature. HCl 6 N (35 μL) was then added followed by the addition of 0.5 mL NaH₂PO₄.H₂O, pH 7.0. HA-BA and HA-IA (5 mg of each) were reacted with activated SAMSA fluorescein for 30 min at room temperature. The reaction mixtures were then separated on an Econo-Pac Bio-Rad column (Bio-Rad Laboratories, Hercules, Calif.) packed with Bio-Gel P-30 Gel with a nominal exclusion limit of 40 kDa (Bio-Rad Laboratories, Hercules, Calif.) to confirm the covalent attachment. To determine the degree of derivatization for haloacetate HAs, the SAMSA fluorescein—haloacetate HA reaction mixture was dialyzed for three days against dH₂O (MWCO 3500) then the A₄₉₄ was spectroscopically determined.

HA Haloacetate Cytotoxicity Assay. T31 human tracheal scar fibroblasts were seeded in 96-well plates at a density of 10⁴ cells/mL (100 μl/well) in DMEMIF12+10% newborn calf serum+2 mM L-glutamine and incubated for 24 h at 37° C./5% CO₂. Stock solutions of 1.5% HABA, HAIA and HA (120 kDa) were prepared in serum free, L-glutamine free growth medium, and the pH of solution was adjusted to 7.5-8 using 0.1 M NaOH. Solutions were then filtered through a 0.45 μm syringe driven filter unit to ensure sterility. The growth medium was then removed and cells were washed twice with 100 μL of serum free, L-glutamine free medium. Working solutions (100 μl of each 1.5%, 1% 0.6%, 0.2% and 0.1% in serum free, L-glutamine free medium) were added onto cells and the plates were further incubated for an additional 24 h. Untreated cells were used as controls. Cell viability was assessed using the reduction of the tetrazolium compound MTS (Cell-Titer 96 Aqueous One Solution Cell Proliferation Assay, Promega, Madison, Wis.) to a colored formazan product. The reduced salt has an absorbance maximum at 490 nm that can be monitored spectrophotometrically and the intensity of the color is proportional to the number of viable cells in the well.

Gelation studies. 1.5% HABA and HAIA solutions were made at pH of 7-8 in 1×PBS buffer. 3% DTPH-modified methylated HA (CHMA) and gelatin-DTPH solutions in 1×PBS pH of 7-8 were used for crosslinking. The synthesis of CHMA is disclosed in International Publication No. WO 2005/056608, which is incorporated by reference. Halogenated HA derivatives and crosslinker (CHMA or gelatin-DTPH, both thiol group containing compounds) solutions, were mixed at different volume ratios (1:1; 1:2; 2:1; 1:3 and 3:1) and set at room temperature. The fastest crosslinking occurred for the gelatin-DTPH/HAIA mix (3:1 volume ratio). High electrophile derived HA content was not suitable for crosslinking. The results are summarized in Table 1.

TABLE 1
Gelation conditions for HA-BA and HA-IA hydrogels.

| | Volume ratios (v/v) | | | | |
|---|---|---|---|---|---|
| | (Molar ratios) | | | | |
| | 1:1 | 1:2 | 2:1 | 1:3 | 3:1 |
| | (1.52:1) | (1:1.32) | (3:1) | (1:2) | (4.5:1) |
| MATERIAL | Gelation times (h) | | | | |
| CHMA/HABA | 24 | — | 9 | — | 9 |
| CHMA/HAIA | 8.5 | 22 | 6.5 | — | 6 |
| Gelatin DTPH/HABA | 8.5 | 45 | 5 | — | 4 |
| Gelatin DTPH/HAIA | 22 | 26 | 5 | 45 | 4 |

The working solutions tested for gelation were: 2% w/v CMHA-S, 2% w/v HABA and 2% w/v HAIA solutions in 1×PBS, pH 7.0, 8.0, 9.0, 10.0, 11.0 and 12.0, adjusted by adding 1 M NaOH. The haloacetate HA containing hydrogels were obtained by using a 3:1 nucleophile to electrophile molar ratio. The CMHA-S only hydrogels (control) were crosslinked through disulfide bonds, by exposure to air. The solution (flowable liquid) to gel (non-flowing hydrogel) transition times were determined by the test tube inversion method. The experiment was repeated three times, with consistent results.

Non-adherent Hydrogels. Hydrogels consisting of CMHA-S and haloacetate HAs were obtained by dissolving 2% w/v solutions of CMHA-S, HABA and HAIA (1×PBS, pH to 9.0) and mixing them in a 3:1 nucleophile to electrophile molar ratio after sterile filtration. The CMHA-S only hydrogels (control) were crosslinked through disulfide bonds, by exposure to air. The composites were then cast in 96 well tissue culture plates and allowed to gel and cure in the hood at room temperature.

Cytoadherent Hydrogels. Cytoadherent hydrogels were obtained by adding thiol-modified gelatin (Gtn-DTPH) to the non-adherent hydrogels described above. Briefly, 2% w/v solution of Gtn-DTPH (1×PBS, pH 9.0) was mixed with 2% w/v CMHA-S (9:1 v/v) then reacted with 2% w/v haloacetate HA solutions (1×PBS, pH 9.0) in a 3:1 nucleophile to electrophile molar ratio after sterile filtration. The CMHA-S and Gtn-DTPH hydrogels (without haloacetate HAs) were crosslinked through disulfide bonds, by exposure to air. As with the non-adherent gels, the composites were cast in 96-well tissue culture plates and allowed to gel and cure in the hood at room temperature. The gelation times for the Gtn-DTPH containing biomaterials were similar to the non-adherent hydrogels.

Hydrogel Cytotoxicity Assay. Tissue culture plates (96-wells) were coated with 50 μl CMHA-S, CMHA-S+HABA, CMHA-S+HAIA, CMHA-S+Gtn-DTPH, CMHA-S+Gtn-DTPH+HABA and CMHA-S+Gtn-DTPH+HAIA hydrogels prepared at pH 9.0 and were allowed to cure overnight in hood. Uncoated wells were used as controls. Gels were then washed three times with 200 μl medium (DMEM/F12+10% newborn calf serum)+2 mM L-glutamine+penicillin/streptomycin), then cells (3.5×10⁴ cells/mL) in the same medium were seeded in each well (100 μl/well). Cells were then incubated for 48 h at 37° C./5% CO₂. The colorimetric assay described above was used to assess the presence of viable cells. Cell attachment was verified microscopically, using an Olympus CKX41 microscope (Olympus America Inc., Melville, N.Y.).

Hydrogel Degradation. To determine the rate of enzymatic degradation of hydrogels in the presence of bovine testicular HAse (225 U/mL), 0.5 mL gels were cast in 17×60 mm glass vials (Fisher Scientific) and allowed to cure overnight. Subsequently, gels were covered with 600 μl 1×PBS, pH 7.4 HAse and placed in an incubator at 37° C. at 150 rpm. At predetermined time intervals 300 μL PBS±HAse was removed and A₂₃₂ values were assessed spectrophotometrically (the absorbance range of oligosaccharides is 200-240 nm). For each time point, the supernatant removed for assaying was replaced with fresh one (±HAse, as required). The absorbance value recorded one day after complete digestion was set as 100% and absorbance values read on previous days were extrapolated to percentages.

Statistical Analysis.

Values, represented as mean ±standard deviation (S.D.) were compared using Student's t-test (2-tailed) with p<0.05 considered statistically significant and p<0.005 or p<0.001 considered highly significant.

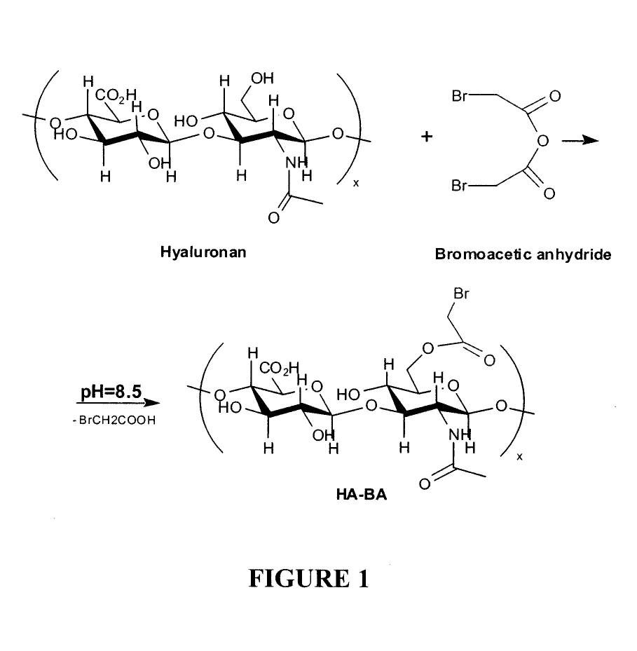

Synthesis and Characterization of Bromoacetate-Derivatized HA (HABA). HABA was obtained by treating HA with bromoacetic anhydride under basic reaction conditions (FIG. 1). The reactant molar excess is needed because of the formation of the mixed anhydride between bromoacetic anhydride and HA, which rapidly hydrolyzes to restore the HA glucuronic acid carboxylic acid groups. While this side reaction would not cause any interference with the overall biological activity of HABA, it consumes the anhydride reagent and reduces the overall bromoacetate modification of the primary hydroxyl groups. Subsequently, the reaction mixture was dialyzed to remove the hydrolyzed bromoacetic anhydride byproducts, sodium bromoacetate and sodium glycolate. The final product (HABA) was obtained at 78% yield by lyophilizing the frozen dialyzed solution.

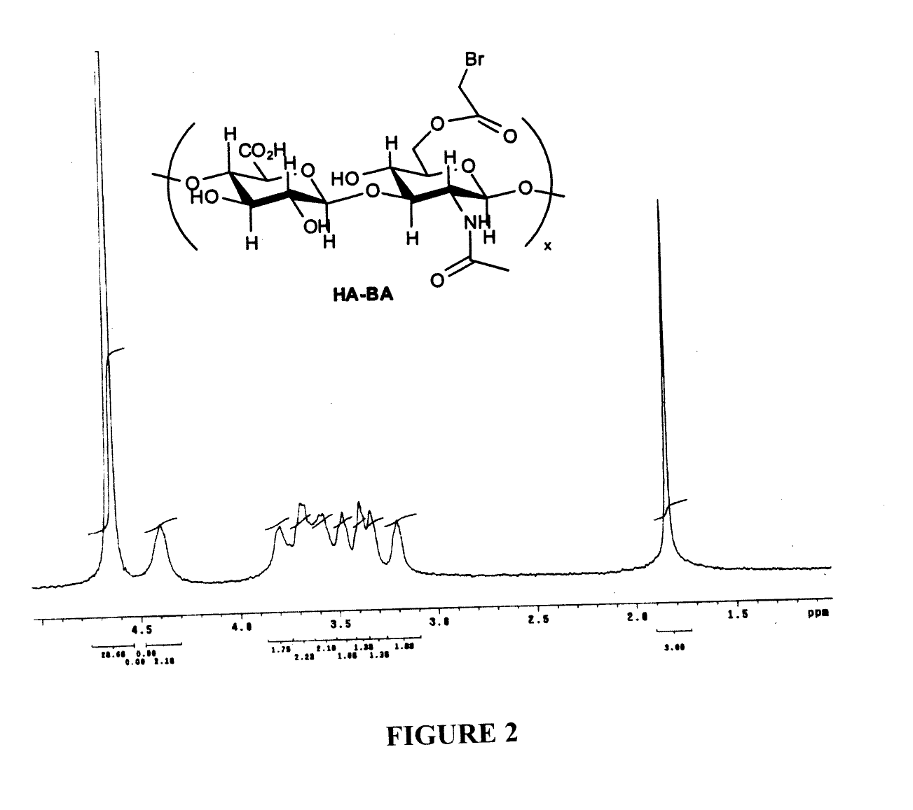

The structure of HABA was confirmed by ¹H-NMR in D₂O. Compared to the spectrum of the starting material (HA) (FIG. 2A), a new broad resonance appeared at 3.84 ppm, corresponding to the methylene protons of the bromoacetate group (COCH₂Br) (FIG. 2B). The purity and molecular weight distribution of HABA were determined by GPC (data not shown). The GPC profile was detected by both refractive index and UV and confirmed the purity of the compound. The molecular weight of the compound was determined to be MW˜120 kDa (polydispersity index 2.58), and the decrease in the molecular weight (compared to the starting material) can be attributed to either basic or acidic hydrolysis during the course of the reaction and purification. The final HABA product is completely soluble in water. The substitution degree, defined as bromoacetate groups per 100 disaccharide units, was estimated by fluorescent dye derivatization to be approximately 18%.

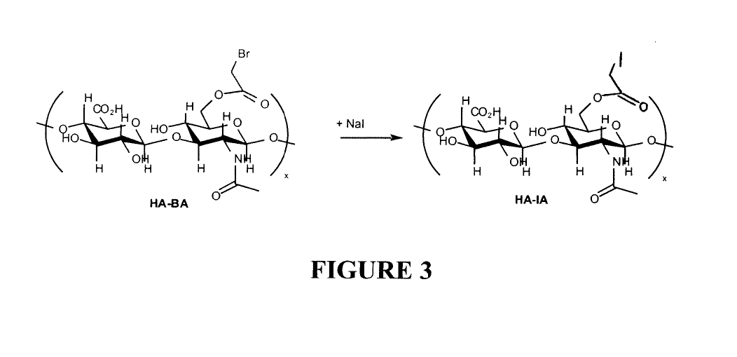

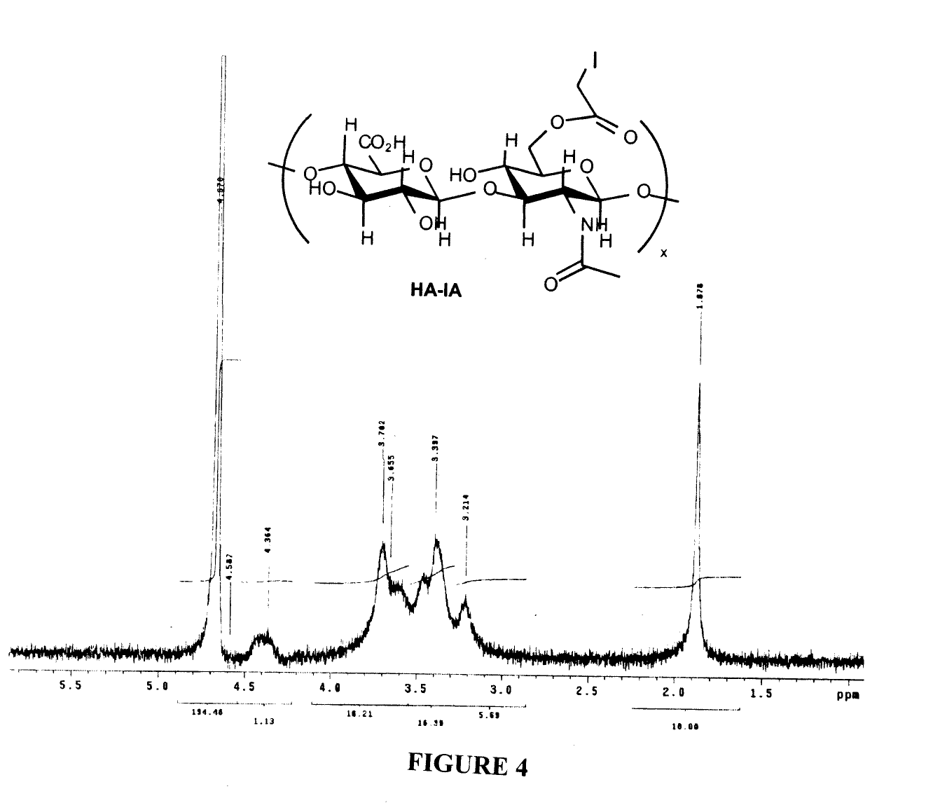

Synthesis and Characterization of Iodoacetate-Derivatized HA (HAIA). HABA obtained was divided into two equal batches. One batch was used for further chemical and biological characterization. The second batch was used as starting material for HAIA synthesis. HABA in nanopure water was reacted with NaI using a modified Finkelstein reaction (FIG. 3), and the solution was dialyzed and lyophilized to give HAIA in 97% yield. Compared to the ¹H-NMR spectrum of the starting HABA (FIG. 2B), the peak corresponding to the methylene protons of the haloacetate group (COCH₂X, δ=3.84) shifted upfield to 3.70 ppm (FIG. 4). GPC was employed to assess the purity and molecular weight distribution of HAIA (data not shown). The molecular weight of the compound was determined to be MW˜160 kDa (polydispersity index 2.45). The substitution degree was presumed to be identical with HABA because of the upfield shift of the ¹H-NMR peak from δ=3.84 to δ=3.70 ppm.

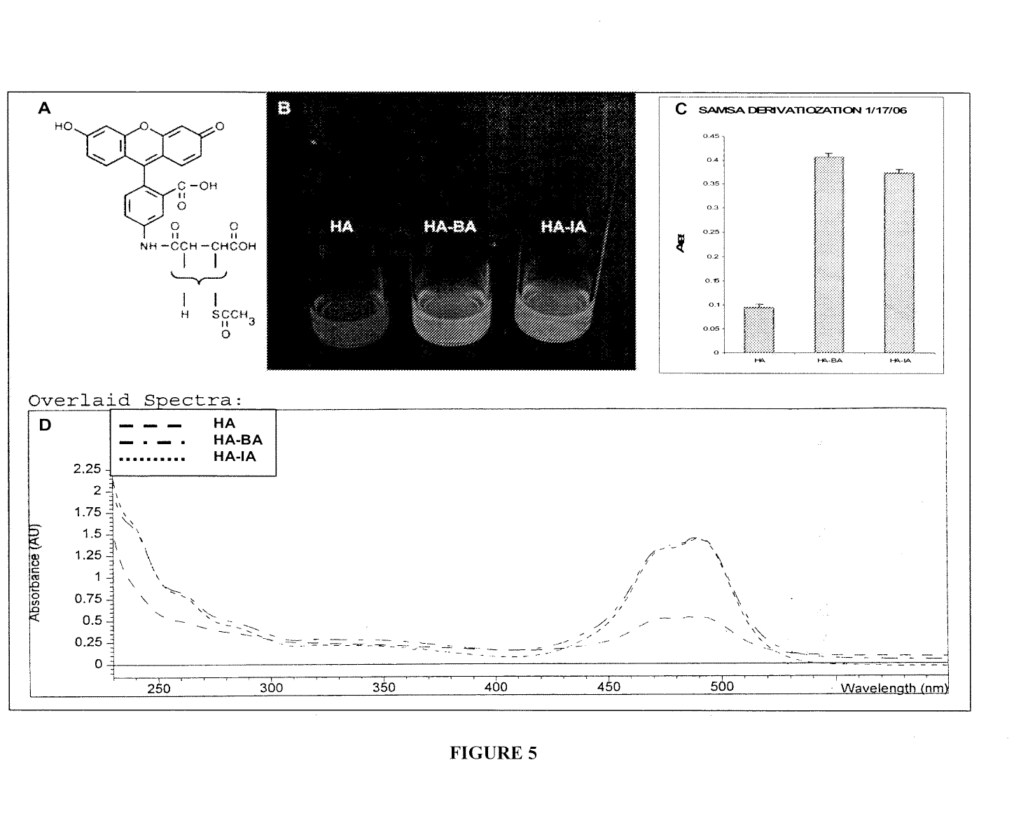

SAMSA Fluorescein Derivatization of HA Haloacetates. The gross structures of the two HA haloacetate derivatives were determined by ¹H-NMR. However, because of the complexity of polymer proton spectra, an additional measure was used to test for successful chemical alteration. SAMSA fluorescein is a thiol group containing fluorescent reagent, commonly used for assaying maleimide and iodoacetamide moieties of proteins (FIG. 5A). Because of the nature of the novel reactive groups, SAMSA fluorescein derivatization was chosen to assess the presence and reactivity of the new moieties (bromoacetate for HABA and iodoacetate for HAIA). After conjugation of HA derivatives with SAMSA fluorescein as described under Materials and Methods and dialysis, the solutions were photographed under UV light to visually assess the fluorescence intensities (FIG. 5B). The covalent attachment of the fluorescent moiety to HA haloacetates was further confirmed chromatographically (results not shown). The results of this experiment represent a proof of concept and show the successful chemical alteration of the HA polymer.

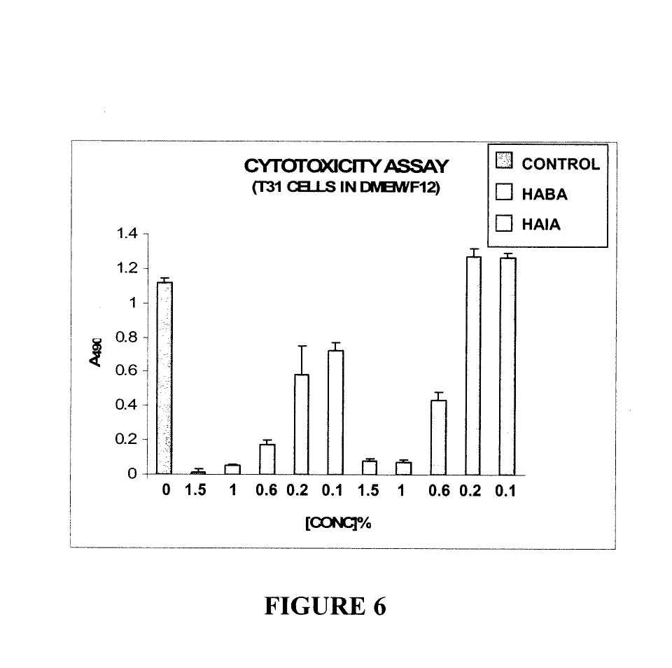

HA Haloacetate Cytotoxicity. Primary human tracheal scar T31 fibroblasts were cultured in 96-well plates and were used as a model system to evaluate the effect of HABA and HAIA on non-immortalized primary cells. The cells were initially cultured in serum containing medium to ensure proper growth. Subsequently, cells were washed with serum free medium, and either HABA or HAIA at w/v concentrations of 1.5%, 1% 0.6, 0.2% and 0.1% in serum-free medium were added then to cells. Cells covered with serum-free medium only were used as controls. After 48 h, cell viability was assessed calorimetrically as described using the MTS assay. As expected for thiol-reactive electrophilic species, the two HA haloacetate polymers were cytotoxic at high concentrations. However, at low concentrations (0.1% w/v), they were well tolerated by these sensitive cells (FIG. 6).

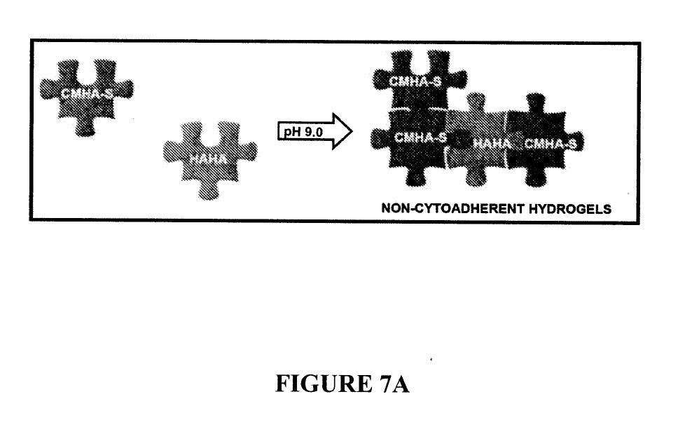

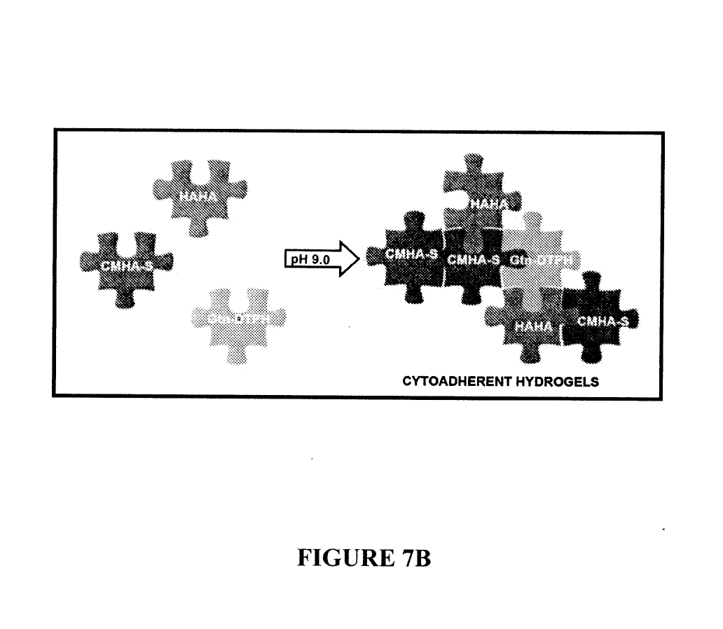

HA Haloacetate Crosslinked Hydrogels. FIG. 7 illustrates the two fundamentally different hydrogels prepared from the HA haloacetates. Thus, FIG. 7A illustrates the preparation of non-cytoadherent hydrogels based exclusively on two chemically-modified HA derivatives—one electrophilic and one nucleophilic. FIG. 7B shows that by incorporation of a thiol-modified gelatin derivative, the electrophilic and nucleophilic HA derivatives can be co-crosslinked into a cytoadherent hydrogel.

To determine the gelation time of haloacetate HA containing biomaterials, hydrogels were prepared by mixing CMHA-S with HABA or HAIA in a 3:1 molar ratio. The pH dependence of gelation times was investigated next. Solutions (2% w/v) of CMHA-S and HABA or HAIA were made in 1×PBS, pH 7.4 and the pH of the solutions was then adjusted to pH 7.0; 8.0; 9.0; 10.0; 11.0 and 12.0. As expected, the fastest setting solutions were those at pH 9.0 and 10.0 (Table 1). The gels obtained were clear and insoluble in aqueous solutions (data not shown). The gelation process of the haloacetate HA containing hydrogels proceeds via a nucleophilic substitution reaction that leads to the formation of a thioether. The thiol groups of CMHA-S have a pKa value of approximately 9, which explains why the optimum pH for the reaction is 9-10. At lower pH values, the thiol group is mostly in its protonated form, while as the pH increases, the relative amount of the anionic nucleophile increases. At pH values above 10, hydroxide begins to displace iodide or bromide, making it unavailable for thioether formation.

Hydrogel Cytotoxicity. The non-cytoadherent hydrogels (FIG. 7A) were prepared by mixing 2% w/v solutions of CMHA-S, pH 9.0 with 2% w/v solutions of HA haloacetates, pH 9.0 in a 3:1 molar ratio. The mixed solutions were then used to coat the wells of a 96-well plate and allowed to gel overnight in the hood. Before cell seeding, the hydrogels were washed serum containing medium then 3.5×10⁴ cells/mL (100 μl/well) were seeded and incubated at 37° C./5% CO₂ for 48 h.

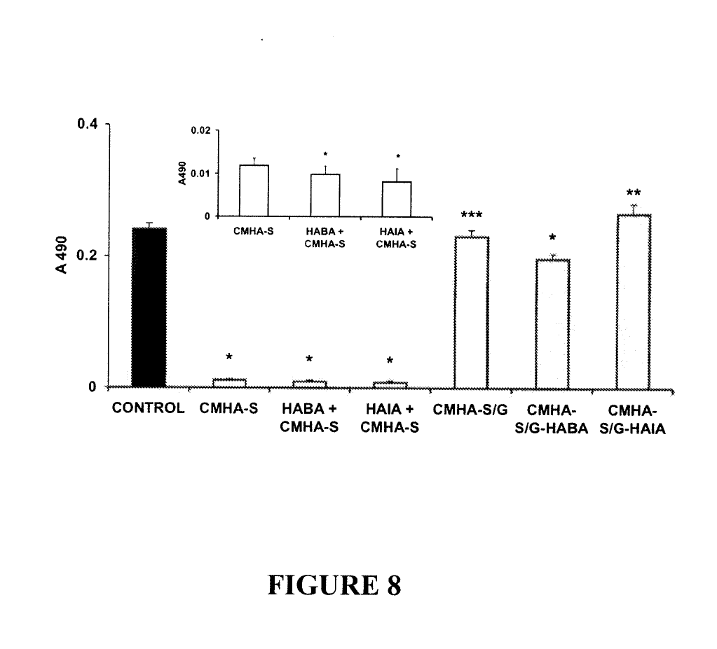

Previous studies show that HA-based hydrogels such as Carbylan™-SX do not promote cell adherence while HA-based gels that contain a covalently-crosslinked gelatin derivative support cell adherence and proliferation. To objectively evaluate the cytotoxicity of haloacetate HA containing hydrogels, uncoated wells and wells coated with covalently-linked gelatin (Gtn-DTPH) containing gels were used as controls (FIG. 7B). After 24 h, cells were examined microscopically. Cells seeded on Gtn-DTPH-free materials, were clustered together, rounded and unattached, while cells grown on plastic only or on Gtn-DTPH-containing materials were spread out and elicited the typical spindle-shape morphology. Cellular viability was assessed by the MTS colorimetric assay 48 h after cell seeding. In the absence of Gtn-DTPH cells were not able to attach. Even fewer cells were present on haloacetate HA containing gels, consistent with the cytotoxic effect of these materials (in hydrogels, the final haloacetate HA concentration is 0.67% w/v because a 2% w/v stock solution is added to the polymer solution at a 1:3 molar ratio) (FIG. 8). Haloacetate HA containing, Gtn-DTPH free, hydrogels with showed 17% (HABA) to 30% (HAIA) decrease in cell adhesion/viability versus the control hydrogels (CMHA-S only) (see FIG. 8 inset).

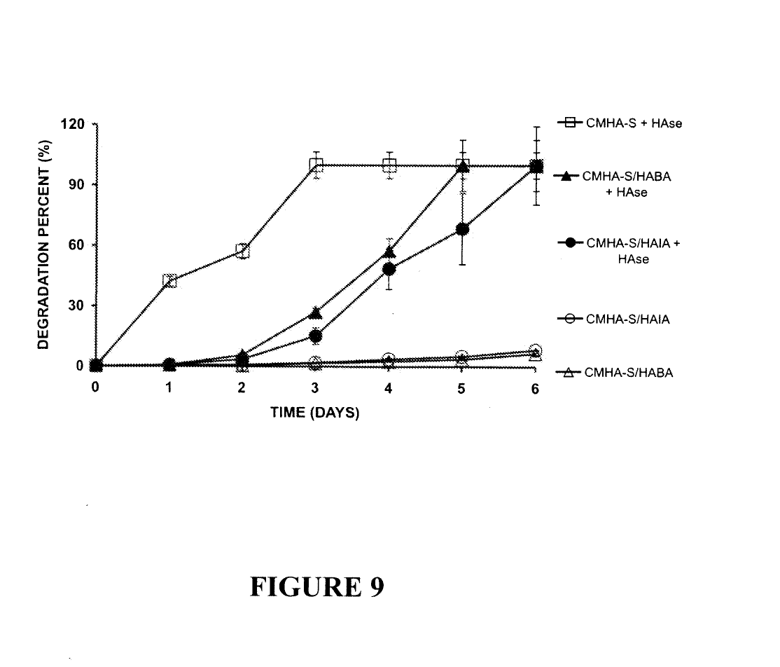

HA Haloacetate Hydrogel Degradation. The use of HA haloacetate hydrogels for medical purposes or any other in vivo application would be dependent on the rate of gel degradation under the action of hyaluronidases which translates to the time that the coating material would actually be present in vivo. To estimate the rate of hydrogel degradation, Gm-DTPH free hydrogels were incubated with 1×PBS, pH 7.4±HAse (225 U/mL). Our results show that CMHA-S hydrogels that are crosslinked via disulfide bonds hydrolyze much faster that the HA haloacetate-containing materials (FIG. 9). By the third day, CMHA-S hydrogels were totally degraded. In contrast, HABA containing hydrogels appeared totally degraded by day 5, while CMHA-S/HAIA hydrogels degraded slightly slower (by day 6). In the absence of enzyme, HA haloacetate-containing hydrogels hydrolyze at a very slow rate. CMHA-S—-only hydrolysis rate could not be determined because this biomaterial has a different behavior that the haloacetate HA containing ones and swells upon supernatant addition.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

What is claimed:

1. A compound comprising the formula I 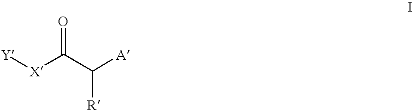
wherein
Y′ is a residue of a macromolecule selected from the group consisting of oligonucleotide, a nucleic acid or a metabolically stabilized analogue thereof, a polypeptide, a glycoprotein, a glycolipid, a polysaccharide and a protein;
X′ is —, —S—, —NH—, or —NR″—;
R′ is hydrogen, alkyl, perfluoroalkyl, aryl, heteroaryl, or halogen;
R″ is hydrogen or C₁₋₅ alkyl; and
A′ is a leaving group.

2. The compound of claim 1, wherein the macromolecule is selected from the group consisting of polysaccharide and a glycosaminoglycan.

3. The compound of claim 2, wherein the polysaccharide comprises hyaluronan, chondroitin sulfate, dermatan, heparan, heparin, dermatan sulfate, heparan sulfate, alginic acid, pectin, chitosan, or carboxymethylcellulose.

4. The compound of claim 1, wherein the macromolecule is selected from the group consisting of synthetic polymer, and the synthetic polymer comprises polyvinyl alcohol, polyethyleneimine, polyethylene glycol, polypropylene glycol, a polyol, a polyamine, a triblock polymer of polypropylene oxide-polyethylene oxide-polypropylene oxide, a star polymer of polyethylene glycol and a dendrimer of polyethylene glycol.

5. The compound of claim 1, wherein the macromolecule is a protein, and the protein is selected from the group consisting of a naturally occurring protein, a recombinant protein, an extracellular matrix protein, a chemically-modified extracellular matrix protein, a genetically engineered protein, and a partially hydrolyzed derivative of an extracellular matrix protein.

6. The compound of claim 1, wherein Y′ comprises a residue of hyaluronan.

7. The compound of claim 1, wherein Y′ comprises a residue of an N-acetyl-glucosamine, wherein at least one primary C-6 hydroxyl group of the N-acetyl-glucosamine residue is substituted with the group —C(O)CH(R′)(A′).

8. The compound of claim 7, wherein at least one secondary hydroxyl group is substituted with the group —C(O)CH(R′)(A′).

9. The compound of claim 7, wherein from 1% of primary C-6 hydroxyl group of the N-acetyl-glucosamine residue to about 100% of the primary C-6 hydroxyl groups of the N-acetyl-glucosamine residue are substituted with the group —C(O)CH(R′)(A′).

10. The compound of claim 1, wherein X′ is —O— or —NH—.

11. The compound of claim 1, wherein R′ is methyl or hydrogen.

12. The compound of claim 1, wherein A′ is a halogen.

13. The compound of claim 1, wherein Y′ is a residue of a hyaluronan, wherein at least one hydroxyl group is substituted with —C(O)CH₂Cl, —C(O)CH₂Br, or —C(O)CH₂₁.

14. A method for making a compound, or a pharmaceutically acceptable salt thereof, comprising reacting a macromolecule comprising at least one nucleophilic group with a compound comprising the formula XV 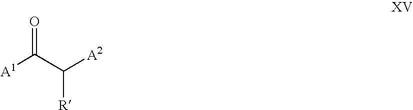
wherein
R′ is hydrogen or an alkyl group; and
A¹ and A² are independently the same or different leaving groups.

15. The method of claim 14, wherein the macromolecule comprises a glycosaminoglycan.

16. The method of claim 14, wherein the macromolecule comprises hyaluronan.

17. The method of claim 14, wherein R′ is hydrogen.

18. The method of claim 14, wherein A¹ forms a compound of the formula XVI 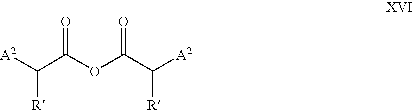
wherein
R′ is hydrogen or an alkyl group, wherein each R′ is the same group; and
each A² is the same leaving group.

19. The method of claim 18, wherein A² is a halogen.

20. The method of claim 18, wherein the macromolecule is hyaluronan and the compound comprising formula XV is selected from the group consisting of chloroacetic anhydride, bromoacetic anhydride, and iodoacetic anhydride.

21. A method for coupling two or more macromolecules, comprising reacting a first macromolecule comprising the formula I in claim 1 with a second macromolecule comprising at least one nucleophilic group.

22. The method of claim 18, wherein the macromolecule is hyaluronan and the compound comprising formula XV is acyl halide, anhydride, or carboxylic acid amide.

23. The method of claim 21, wherein the second macromolecule is selected from the group consisting of an oligonucleotide, a nucleic acid or a metabolically stabilized analogue thereof, a polypeptide, a glycoprotein, and a glycolipid.

24. The method of claim 21, wherein the second macromolecule comprises a polysaccharide having at least one SH group.

25. The method of claim 21, wherein the second macromolecule comprises a glycosaminoglycan having at least one SH group.

26. The method of claim 21, wherein the second macromolecule is selected from the group consisting of chondroitin sulfate, dermatan, heparan, heparin, dermatan sulfate, heparan sulfate, alginic acid, pectin, chitosan, carboxymethylcellulose, and hyaluronic acid having at least one SH group.

27. The method of claim 21, wherein the second macromolecule comprises the formula II 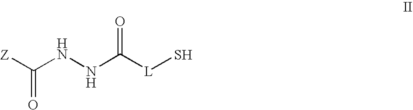
wherein
Z is a residue of a macromolecule, and
L is selected from the group consisting of a polyalkylene group, a polyether group, a polyamide group, a polyamino group, an aryl group, a polyester, and a polythioether group.

28. The method of claim 27, wherein the macromolecule is selected from the group consisting of an oligonucleotide, a nucleic acid or a metabolically stabilized analogue thereof, a polypeptide, a glycoprotein, a glycolipid, a polysaccharide, a protein, and a glycosaminoglycan,
or a pharmaceutically-acceptable compound.

29. The method of claim 27, wherein Z is a residue of hyaluronan and L is CH₂CH₂ or CH₂CH₂CH₂.

30. The method of claim 27, wherein Z is a residue of gelatin and L is CH₂CH₂ or CH₂CH₂CH₂.

31. The method of claim 21, wherein the second macromolecule comprises the formula XX
Y—X—R—SH XX
wherein
Y is a residue of a macromolecule;
X is —O—, —S—, —NH—, or —NR″;
R″ is hydrogen or C₁₋₅ alkyl; and
R is a substituted or unsubstituted C₂ or C₃ alkylene group.

32. The method of claim 31, wherein the macromolecule is selected from the group consisting of an oligonucleotide, a nucleic acid or a metabolically stabilized analogue thereof, a polypeptide, a glycoprotein, a glycolipid, a polysaccharide, a protein, and a synthetic polymer, glycosaminoglycan, or a pharmaceutically-acceptable compound.

33. The method of claim 32, wherein the polysaccharide is selected from the group consisting of chondroitin sulfate, dermatan, heparan, heparin, dermatan sulfate, heparan sulfate, alginic acid, pectin, chitosan, hyaluronan, or carboxymethylcellulose.

34. The method of claim 31, wherein the macromolecule is selected from the group consisting of a synthetic polymer, and the synthetic polymer comprises polyvinyl alcohol, polyethyleneimine, polyethylene glycol, polypropylene glycol, a polyol, a polyamine, a triblock polymer of polypropylene oxide-polyethylene oxide-polypropylene oxide, a star polymer of polyethylene glycol and a dendrimer of polyethylene glycol.

35. The method of claim 31, wherein the macromolecule is a protein selected from the group consisting of a naturally occurring protein, a recombinant protein, an extracellular matrix protein, a chemically-modified extracellular matrix protein, a genetically engineered protein, and a partially hydrolyzed derivative of an extracellular matrix protein.

36. The method of claim 31, wherein X is —O— or —NH—.

37. The method of claim 31, wherein R is CH₂CH₂, CH₂CH₂CH₂, CH₂CHR⁵, CHR⁵CHR⁵, C(R⁵)₂CHR⁵, or C(R⁵)₂C(R⁵)₂, wherein R⁵ is an alkyl group.

38. The method of claim 31, wherein R is CH₂CH₂.

39. The method of claim 31, wherein Y is a residue of a hyaluronan, wherein at least one hydroxyl group is substituted with —CH₂CH₂SH.

40. A compound made by the method of claim 21.

41. A compound, or a pharmaceutically acceptable salt thereof, having at least one fragment comprising the formula VII 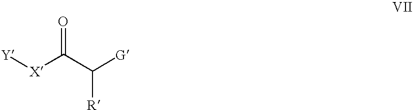
wherein
Y′ is a residue of a first macromolecule;
X′ is O—, —S—, —NH—, or —NR″—;
R′ is hydrogen or an alkyl group;
R″ is hydrogen or C₁₋₅ alkyl; and
G′ comprises a residue of a second macromolecule.

42. A pharmaceutical composition comprising a pharmaceutically-acceptable compound and one or more compounds of claim 1.

43. A pharmaceutical composition comprising a pharmaceutically-acceptable compound and one or more compounds of claim 41.

44. A pharmaceutical composition comprising a living cell and one or more compounds of claim 1.

45. A pharmaceutical composition comprising a living cell and one or more compounds of claim 41.

46. A method for improving wound healing in a subject in need of such improvement, comprising contacting the wound of the subject with one or more compounds of claim 1.

47. A method for improving wound healing in a subject in need of such improvement, comprising contacting the wound of the subject with one or more compounds of claim 41.

48. A method for delivering at least one pharmaceutically-acceptable compound to a patient in need of such delivery, comprising contacting at least one tissue capable of receiving the pharmaceutically-acceptable compound with the composition of claim 42.

49. The use of the compound of claim 1 as a growth factor, an anti-inflammatory agent, an anti-cancer agent, an analgesic, an anti-infective agent, or an anti-cell attachment agent.

50. The use of the compound of claim 41 as a growth factor, an anti-inflammatory agent, an anti-cancer agent, an analgesic, an anti-infective agent, or an anti-cell attachment agent.

51. A substrate comprising one or more compounds of claim 1.

52. A substrate comprising one or more compounds of claim 41.

53. The substrate of claim 51, wherein the substrate comprises a laminate, a gel, a bead, a sponge, a film, a mesh, an electrospun nanofiber, a woven mesh, or a non-woven mesh.

54. A method for growing a plurality of cells, comprising (a) depositing a parent set of cells on the substrate of claim 51, and (b) culturing the substrate with the deposited cells to promote the growth of the cells.

55. A method for growing cells, comprising contacting the cells with one or more compounds of claim 1.

56. A method for growing cells, comprising contacting the cells with one or more compounds of claim 41.

57. The method of claim 54, wherein the cell comprises a stem cell.

58. The method of claim 55, wherein the cell comprises a stem cell.

59. A method for repairing a damaged elastic tissue in a subject, comprising contacting the damaged tissue with one or more compounds of claim 1.

60. A method for repairing a damaged elastic tissue in a subject, comprising contacting the damaged tissue with one or more compounds of claim 41.

61. The method of claim 58, wherein the tissue comprises a vocal cord, a cardiovascular tissue, a muscle, a tendon, a ligament, bladder tissue, tissue in the urethra, a sphincter muscle, or a muscle in the gastrointestinal tract.

62. A bio-artificial material comprising the reaction product between one or more compounds of claim 1 and a macromolecule comprising at least two thiol groups.

63. A bio-artificial material comprising the reaction product between one or more compounds of claim 41 and a macromolecule comprising at least two thiol groups.

64. The bio-artificial material of claim 62, wherein the macromolecule comprises an elastin-like peptide with at least two thiol groups.

65. A prosthetic device comprising one or more bio-artificial materials of claim 62.
